(19) United States
(12) Patent Application Publication
(10) Pub. No.: US 2009/0162919 A1
(43) Pub. Date: Jun. 25, 2009

(54) METHODS FOR CONCENTRATING MICROALGAE

(75) Inventors: Guido Radaelli, Oakland, CA (US); Daniel Fleischer, Oakland, CA (US); Bertrand Vick, Berkeley, CA (US); Matthew Caspari, San Francisco, CA (US); Joseph Weissman, Vero Beach, FL (US); David Rice, Vero Beach, FL (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Aurora Biofuels, Inc., Alameda, CA (US)

(21) Appl. No.: 12/339,521

(22) Filed: Dec. 19, 2008

Related U.S. Application Data
(60) Provisional application No. 61/016,387, filed on Dec. 21, 2007.

(51) Int. Cl. C12N 1/12

(52) U.S. Cl. 435/257.6; 435/257.1

(57) ABSTRACT

The present invention provides commercially viable, large-scale methods for concentrating microalgae with an average diameter of about 20 μm or less. The methods find use in concentrating microalgae with an average diameter of about 5 μm or less, for example, Nannochloropsis.

### CROSS-REFERENCES TO RELATED APPLICATIONS

The present application claims the benefit of U.S. Provisional Application No. 61/016,387, filed on Dec. 21, 2007, the entire of disclosure of which is hereby incorporated herein by reference for all purposes.

### FIELD OF THE INVENTION

The present invention relates to the field of concentrating and harvesting microalgae.

### BACKGROUND OF THE INVENTION

Microalgae differentiate themselves from other single-cell microorganisms in their natural ability to accumulate large amounts of lipids. For example, the Aquatic Species Program conducted by NREL from mid-70s to mid-90s identified about 300 species of microalgae suitable for oil production (“A look back to the Aquatic Species Program”, Sheehan J., Dunahay T., Benemann J. R., Roessler P., 1996, NREL/TP-580-24190). All lipidic compounds have the potential to generate biofuels and renewable energy. However, triglycerides are of particular importance for the production of biodiesel via transesterification, a process commonly used for the conversion of vegetable oil from canola, soy, corn, sunflower, and palm into biodiesel.

Microalgal lipids are also known to contain fatty acids especially valuable as dietary supplements, including omega-3s and omega-6s. Among these omega-3 and omega-6 compounds, EPA (EicosaPentaenoic Acid) and DHA (DocosaHexaenoic Acid) are commercially valuable and currently marketed in several different formulations as dietary supplements for adults, health supplements in infant nutritional products, and additives to animal feed. For example, Schizochytrium has been demonstrated to produce high levels of DHA when cultured heterotrophically in sterile fermentors (“Heterotrophic production of long chain omega-3 fatty acids utilizing algae and algae-like microorganisms”, Barclay W. R., Meager K. M., Abril J. R., Journal of Applied Phycology (1994) 6(2)123-129) while Nannochloropsis is able to accumulate high concentrations of EPA if cultured autotrophically in open ponds or closed photobioreactors, especially if starved for nitrogen nutrients (“Chemical profile of selected species of microalgae with emphasis on lipids”, Ben-Amotz A., Tornabene T. G., Thomas W. H. Journal of Phycology (1985) 21(1) 72-81; Sukenik, “Production of eicosapentaenoic acid by the marine eustigmatophyte Nannochloropsis” in Chemicals from Microalgae, Cohen, Z., ed. 1999, pp. 41-56; “Production of eicosapentaenoic acid by Nannochloropsis sp. cultures in outdoor tubular photobioreactors”; Chini Zittelli G., Lavista F., Bastianini A., Rodolfi L., Vincenzini M., Tredici M. R., Journal of Biotechnology (1999) 70(1-3):299-312).

Microalgae are also a useful source of carotenoids. Astaxanthin, lutein, beta-carotene and other carotenoids, all present in several species of microalgae, represent as a whole an approximately billion dollar world market. For example, Dunaliella is mass cultured in open ponds for the industrial production of natural beta-carotene (U.S. Pat. No. 4,199,895) and Haematococcus is cultivated for the production of astaxanthin, a valuable anti-oxidant used as food supplement (U.S. Pat. No. 6,022,701).

Among all microalgal genera, Nannochloropsis has a unique potential for commercial scale-up in that it can be a natural source of lipids for fuel production, of omega-3s for dietary supplements and animal feed, and of carotenoids such as violaxanthin, which is important in the poultry industry given its role in the egg yolk pigmentation (“Enrichment of poultry products with ω3 fatty acids by dietary supplementation with the alga Nannochloropsis and mantur oil”, Nitsan Z., Mokady S., Sukenik A., J. Agric. Food Chem. (1999) 47(12), 5127-5132).

The process of mass culturing all commercial microalgal strains is characterized by high production costs, which, up to date, has restricted the microalgae industry to the production and sale of high value niche products, including nutraceuticals, pharmaceuticals and cosmetics. The production of microalgal biomass is very expensive for two main reasons: i) cultivating microalgae in raceway ponds and in closed photobioreactors requires large capital investment and has significant operating costs; ii) harvesting microalgal biomass from aqueous culture is extremely difficult for most strains (Spirulina being a notable exception), and it requires considerable investment in equipment and significant energy consumption.

Harvesting the microalgal biomass is very difficult and expensive because i) the biomass density in the culture is usually very low, e.g., 200-300 mg/l in open ponds, up to 2,000 mg/l in closed photobioreactors, and ii) most microalgae are single-cell free floating organisms—the cell size varies typically between 5 and 30 μm-without any natural tendency to aggregate in colonies. In view of these characteristics, the only process that has proven to be reliable for the industrial application is centrifugation. In fact, centrifuges are normally utilized in the production of beta-carotene from Dunaliella, of astaxanthin from Haematococcus, of omega-3 rich biomass and oil from Nannochloropsis, of dietary supplements from Chlorella, and of aquaculture feedstock from several strains. Centrifugation has extremely high capital and operating costs and is one of the critical cost drivers in any current microalgal industrial process, thus preventing the algae industry from obtaining access to lower value and higher volume products (“The potential of new strains of marine and inland saline-adapted microalgae for aquaculture applications”, Barclay W., Terry K., Naigle N., Weissman J., Goebel R. P., J. World Aquaculture Soc. (1987) 18:216-228).

The only microalgal process where centrifugation is not utilized is Spirulina cultivation. Spirulina, in fact, naturally grows in filamentous colonies which allow the use of simple and inexpensive filtration methods, such as automatic micro-screening or manual filtration with cloth-filters. Due to this particular characteristic, Spirulina production has the lowest production cost among all microalgae processes.

In contrast, methods applied to concentrating and separating larger microalgae have not been applied with success to very small microalgae (i.e., microalgae having a diameter of about 10 μm or less), for example, Nannochloropsis. See, e.g., Knuckey R. M., Brown M. B., Robert R., Frampton D. M. F, Aquacultural Engineering (2006) 35(3):300-313; and Lubian L., Aquacultural Engineering (1989) 8(4):257-281). Accordingly, there remains a need for separating and concentrating very small microalgae in a commercially viable manner.

### BRIEF SUMMARY OF THE INVENTION

The present invention provides economically viable and industrial-scale methods and compositions for the flocculation of microalgae that do not spontaneously aggregate in colonies or flocs (e.g., microalgae with an average diameter of about 10 μm or less, for example of about 5 μm or less) using low concentrations of organic flocculant (e.g., less than 10% of the dry weight of biomass or less than about 100 mg/l). It will be appreciated that the present methods can be utilized for any single-cell free floating microorganism, and it has been surprisingly found that the present methods allow for the efficient concentration and separation of microalgae from the genus Nannochloropsis. Following flocculation, the microalgae are concentrated, for example, by air flotation or by sedimentation. The concentrated algal biomass can be optionally further concentrated via filtration or centrifugation and the resulting sludge can be further processed for biofuels, animal feed, dietary supplements, fertilizer, cosmetic and pharmaceutical products, or directly used as aquaculture feedstock.

Accordingly, in one aspect, the invention provides methods of concentrating single cell microalgae in an aqueous environment. In some embodiments, the methods comprise:

a) contacting microalgae having an average single cell diameter of less than 20 μm, for example, less than 15 μm, 10 μm or 5 μm, in an aqueous environment with an inorganic flocculant present at a concentration that is less than 20%, for example, less than 10%, of the dry biomass of the microalgae, thereby yielding flocculated microalgae in flocs having an average diameter of at least 100 μm; and

b) separating the flocs of microalgae from the aqueous environment, thereby concentrating the microalgae into a slurry with a biomass density of at least 1%.

In some embodiments, the inorganic coagulant is present at a concentration of 100 mg/l or less. In some embodiments, the flocculant is present at a concentration between 2-80 mg/l, for example, 10-60 mg/l, 5-15 mg/l, 2-10 mg/l, 3-8 mg/l, 4-7 mg/l or 2-5 mg/l. In some embodiments, the flocculant is present at a concentration of less than about 10 mg/l, for example less than about 5 mg/l, for example, 10 mg/l, 9 mg/l, 8 mg/l, 7 mg/l, 6 mg/l, 5 mg/l, 4 mg/l, 3 mg/l or 2 mg/l.

In some embodiments, the flocculant is an iron flocculant or an aluminum flocculant. In some embodiments, the flocculant is an aluminum flocculant selected from the group consisting of aluminum chloride, aluminum sulfate, polyaluminum chloride, aluminum chlorohydrate, and sodium aluminate. In some embodiments, the flocculant is an iron flocculant selected from the group consisting of ferric chloride, ferric sulfate, and ferrous sulfate.

In some embodiments, the flocculant is not algicidal.

In some embodiments, the microalgae are in a non-natural body of water.

In some embodiments, the microalgae in the aqueous environment are essentially a monoculture.

In some embodiments, the flocs of microalgae are separated from the aqueous environment and concentrated to produce a slurry with a biomass density of about 1-10%, for example, about 2%, 3%, 4%, 5%, 6%, 7%, 8%, 9% or 10%.

In some embodiments, the separating step comprises subjecting the flocculated algae to air flotation.

In some embodiments, the separating step comprises subjecting the flocculated algae to sedimentation.

In some embodiments, the microalgae is from a microalgal strain selected from the group consisting of Dunaliella, Chlorella, Tetraselmis, Botryococcus, Haematococcus, Phaeodactylum, Skeletonema, Chaetoceros, Isochrysis, Nannochloropsis, Nannochloris, Pavlova, Nitzschia, Pleurochrysis, Chlamydomas and Synechocystis.

In some embodiments, the microalgae is Nannochloropsis.

In some embodiments, the methods further comprise the step of contacting the microalgae with an organic polymer. In some embodiments, the organic polymer is a cationic or a non-ionic polymer. In some embodiments, the organic polymer is comprised of monomers selected from the group consisting of acrylamide, acrylate, amine or mixtures thereof. In some embodiments, the organic polymer is from a naturally occurring source. For example, the organic polymer can be chitosan or a clay. In some embodiments, the clay is a phosphatic clay, for example, comprising one or more minerals selected from montmorillonite, palygorskite, phosphorite, kaoline, yellow loess, and mixtures thereof. In some embodiments, the organic polymer is present in a concentration of less than 2% of the weight of the dry biomass.

In some embodiments, the aqueous environment is free of sewage. In some embodiments, the aqueous environment is free of polybasic carboxylic acid. In some embodiments, the aqueous environment contains only trace amounts of copper.

In some embodiments, the aqueous environment is less than pH 10. In some embodiments, the aqueous environment is between pH 7-9. In some embodiments, the aqueous environment is not externally pH adjusted.

In some embodiments, the aqueous environment has a salinity of at least 20 ppt.

### DEFINITIONS

The term “microalgae” refers to microphytes, e.g., unicellular eukaryotic species that exist individually or in chains or groups. The microalgae subject to the present concentrating methods generally have an average diameter of about 20 μm or less, for example, about 15 μm, 10 μm, 5 μm, or less. In some embodiments, the microalgae are photosynthetic algae. In some embodiments, the microalgae are of the genus Dunaliella, Chlorella, Tetraselmis, Botryococcus, Haematococcus, Phaeodactylum, Skeletonema, Chaetoceros, Isochrysis, Nannochloropsis, Nannochloris, Pavlova, Nitzschia, Pleurochrysis, Chlamydomas or Synechocystis.

The terms “coagulant” or “flocculant” interchangeably refer to any compound or substance that promotes coagulation or flocculation, i.e. the process of contact and adhesion whereby individual cells of a dispersion form clusters of two or more cells (e.g., floes).

A “floc” refers to a cluster of two or more cells formed in the flocculation process. The floc formed by the present methods can have an average diameter of at least about 100 μm, for example, about 150 μm, 200 μm or 250 μm, 500 μm, 1000 μm, or larger. In some embodiments, flocs formed by the present methods can be composed of at least 10², 10¹, 10⁴, 10⁵ cells, or more.

The term “organic polymer” refers to any organic polymeric compound, i.e. a chemical substance whose structure comprises a long sequence of monomers. The organic polymer can be synthetic or naturally occurring.

The terms “aqueous environment” or “aqueous mixture” or “aqueous culture” interchangeably refer to a liquid environment or mixture or culture, wherein the liquid is at least 50% water. In some embodiments, the aqueous mixture or aqueous environment is brackish or has a salinity equivalent to sea water. For example, in some embodiments, the aqueous mixture or aqueous environment has a salinity of at least about 20 parts per thousand (ppt), for example, at least about 25 ppt, 30 ppt, 35 ppt, or 40 ppt. Expressed another way, the aqueous mixture or aqueous environment can have an ionic strength of at least about 0.5, for example, at least about 0.6, 0.7 or 0.8. The aqueous mixture or aqueous environment can have a naturally occurring, i.e., occurring without adding further acid or base, pH of about 7-10, for example of about 7.5-8.5, or about 8.0-9.0, or about 7.0, 7.5, 8.0, 8.5, 9.0, 9.5, or 10.0.

The phrases “non-naturally occurring” or “unnatural” body of water interchangeably refer to any body of water which is contained in an artificial basin filled with water. The water can come from any source, including an ocean, a sea, a lake or a river. The body of water can be open (e.g., uncovered, outside, for example, in a raceway pond) or enclosed (e.g., in a controlled growth tank, for example, a photobioreactor). The body of water can be any volume.

“Density” refers to the amount of solids (biomass) in an aqueous solution or slurry. Density can be defined as grams of biomass (dry basis) per liter of solution/slurry. Biomass density (dry basis) can be determined through a dry weight analysis, as set forth in the assay for determination of culture biomass concentration, below.

“Dissolved Air Flotation” or “DAF” refers to the method of separating particles or cells from a liquid mixture by causing the particles or cells to collect on the surface of air bubbles suitably dissolved in the mixture itself.

The phrase “consisting essentially of” refers to the elements expressly set forth and can include non-essential or incidental elements, but excludes other active elements not expressly mentioned. For example, in some embodiments, the aqueous mixtures, aqueous environment or culture will be free of or only include trace (e.g., less than can be detected using standard methods or less than about 1 mg/L, for example, less than about 1 μg/L or 1 ng/L) amounts of copper, polybasic carboxylic acids, sewage, or algacides.

The term “large-scale” refers to commercial scale or industrial scale applications of the methods. In some embodiments “large-scale” production of microalgae refers to a culture of at least about 100 L, for example, at least about 200, 400, 500, 750, or 1000 L, for example, at least about 5000, 8000, 10000, 15000, 20000 L, or more.

The term “monoculture” refers to the culture of one species of microorganism (e.g., microalgae) in an aqueous mixture or environment. In some embodiments, a monoculture will have less than 10% contamination, for example, less than 8%, 5%, 3%, 2%, or 1% contamination, with microorganisms not being grown or cultured in the monoculture (i.e., the aqueous mixture contains essentially a monoculture of the microorganism intended to be cultured).

### DETAILED DESCRIPTION

### 1. Introduction

The present invention provides methods and compositions for large-scale and economically viable flocculation and concentration of single-cell free floating microalgae with an average diameter of less than about 10 μm, for example, less than about 5 μm (e.g., Nannochloropsis), using low concentrations of organic flocculant. This process provides economic viability to the mass generation of algal biomass, which is the intermediate in the production of algal based products, including biofuels, food supplements, nutraceuticals, animal feed supplements, and products for the cosmetic and pharmaceutical industry.

### 2. Methods

The processes of the invention can be practiced with any engineered, bred, or naturally occurring microorganism that is characterized by a size of about 20 μm or less, for example, about 15 μm, 10 μm, 5 μm or less. Microorganisms of this size generally do not aggregate in colonies, floes or filaments and do not spontaneously settle to the bottom or float to the surface, but instead are free floating in the culture medium. Further, the microorganism may include fungi, such as yeast, or microorganisms such as bacteria or unicellular algae. In some embodiments, the organism is an algal organism, for example, a photosynthetic microalgae or a green microalgae. In some embodiments, the microalgae are of a spherical shape. Exemplified microalgae include those from a microalgal strain of the genus Dunaliella, Chlorella, Tetraselmis, Botryococcus, Haematococcus, Phaeodactylum, Skeletonema, Chaetoceros, Isochrysis, Nannochloropsis, Nannochloris, Pavlova, Nitzschia, Pleurochrysis, Chlamydomas or Synechocystis. In some embodiments, the microalgae from the microalgal phyla Eustigmatophyceae, Chlorophyceae, or Prasinophyceae. In some embodiments, the algae are of the genus Nannochloropsis.

The starting concentration of the microalgae in the culture can be in the range of about 100 mg/l to about 2000 mg/l, for example, about 200 mg/l, 250 mg/l, 300 mg/l, 500 mg/l, 1000 mg/l, 1500 mg/l or 2000 mg/l.

The present invention provides methods and compositions for separating single cell free floating organisms from their culture and concentrating them in an aqueous sludge or slurry having a biomass density of at least 1%, for example, 1-10%, or more. The present invention is particularly suitable for the flocculation of microalgal organisms, whose harvesting methods from the growth culture are currently very expensive and not economically feasible for low value (e.g., a value below $1,000/ton) and large volume products like biofuels. In one embodiment, the invention relates to a process whereby a culture comprising single cell free floating microalgae is flocculated by adding an inorganic coagulant in a concentration that is less than 20%, for example, less than 10%, of the weight of the dry biomass. The inorganic coagulant can be dissolved in the aqueous mixture at a concentration that is about 100 mg/l or less, for example, ranging between about 2 and 100 mg/l, for example, about 2-80 mg/l, for example, 10-60 mg/l, 5-15 mg/l, 2-10 mg/l, 3-8 mg/l, 4-7 mg/l or 2-5 mg/l, into the algal culture, stirring the culture to promote the contact between the flocculant and the microorganisms, and letting the microorganisms aggregate into flocs of at least about 100 μm. In some embodiments, the inorganic coagulant is present at a concentration of less than about 10 mg/l, for example less than about 5 mg/l, for example, 10 mg/l, 9 mg/l, 8 mg/l, 7 mg/l, 6 mg/l, 5 mg/l, 4 mg/l, 3 mg/l or 2 mg/l.

An organic polyelectrolyte or polymer, can be further added in a concentration that is less than about 2% of the weight of the dry biomass to produce the aggregation of the coagulated flocs into larger flocs. Larger flocs, with a size in the order of millimeter (mm), can be generated if an organic polyelectrolyte is added to the coagulated solution. The polymer can be synthetic or natural. Usually, the polymer will be cationic or non-ionic. In some embodiments, the organic polymer is a polyacrylamide, a polyacrylate, a polyamine or a co-polymer comprising two or more of acrylamide, acrylate and amine monomers. Polymers with a molecular weight of is about 5000 daltons or less, for example about 4000, 3000, 2000, 1000, 800 daltons, or less, find use. Polymers suitable for use in the present methods include, without limitation, Tramfloc T141, Zetag 8818, Praestol K290FL and Monolyte 6016.

In some embodiments, the organic polymer is derived from a naturally occurring material, for example, chitosan or a clay. In some embodiments, the clay is a phosphatic clay, for example, comprising one or more minerals selected from montmorillonite, palygorskite, phosphorite, kaoline, yellow loess, and mixtures thereof. See, e.g, Beaulieu, et al., Harmful Algae (2005) 4:123-138; and Sengco and Anderson, J Eukaryot Microbiol (2004) 51(2):169-172.

Once cells are aggregated in flocs of a size of 100 μm or larger, their separation can be performed using any method for concentration and/or removal known in the art, including but not limited to sedimentation, air flotation, centrifugation, and filtration, including belt filtration, cross filtration, tangential filtration, and press filtration.

Air flotation or sedimentation can be used to concentrate and remove the flocs from the aqueous solution and generate a biomass slurry with a density of at least about 1%, for example, about 1-10%, or more. In some embodiments, at least 70% of the biomass is in the recovered sludge (i.e., biomass slurry); i.e., no more than 30% of the biomass is left in the clarified solution.

Flocculating single cell free floating microorganisms, in particular microalgae, that do not spontaneously aggregate in colonies, has always been a challenge, especially if the flocculation has to be achieved with a very low cost of production. Numerous studies have shown that traditional inorganic flocculants, including alum, ferric chloride, ferrous sulphate and lime, are effective for algae flocculation and removal only at economically non-viable concentrations, for example, higher than 100 mg/l, oftentimes as high as 200 and 300 mg/lI. Furthermore, the particular operating conditions that they require to be effective, particularly high pH, make the usage of inorganic flocculants extremely expensive (“Harvesting and processing sewage-grown planktonic algae”, Golueke G. C., Oswald W. J., Journal of the Water Pollution Control Federation (1965) 37:471-498; Benemann J. R., Koopman B. L., Weissman J. C., Eisenberg D. E., “Development of microalgae harvesting and high-rate pond technology” in Algae Biomass, 1980, Shelef and Soeder, Elsevier/North Holland Biomedical Press, pp 457-493; “Flocculation of microalgae in brackish and sea waters”, Sukenik A., Bilanovic D., Shelef G., Biomass (1988) 15(3):187-199.

More recently organic polymers, including chitosan and a score of branded proprietary organic flocculating agents, have been tested on microalgae. For example, chitosan, Zetag 63 and CF 400 were effective for the harvesting of Chlorella, a freshwater species, only at concentrations above 10 mg/l (“Evaluation of various flocculants for the recovery of algal biomass grown on pig-waste”, Buelna G., Bhattarai K. K., de la Noue J., Taiganides E. P., Biological Wastes BIWAED (1989) 31(3):211-222; “Flocculation of algae using chitosan”, Divakaran R., Sivasankara Pillai V. N., Journal of Applied Phycology (2002) 14(5):419-422. Flocculating marine algae grown in seawater (salinity in the order of 20-50 parts per thousand (ppt)) is generally more difficult, requiring concentrations of chitosan in the range of 10 to 100 mg/l for a >80% biomass removal (“Concentrating cultured marine microalgae with chitosan”, Lubian L., Aquacultural Engineering (1989) 8(4):257-281). Only a maximum chitosan concentration of 1-3 mg/l would be acceptable from a cost standpoint for the production of large volume products with a value below $ 1,000/ton.

The effectiveness of a flocculant can depend on the specific strain of microalgae. For example, species of the genus Nannochloropsis have been proven to be very difficult to flocculate because Nannochloropsis is spherical and particularly small (about 3-5 μm average diameter), and therefore requires considerably higher concentrations of flocculants than most other algal genera (“Production of microalgal concentrates by flocculation and their assessment as aquaculture feeds”, Knuckey R. M., Brown M. B., Robert R., Frampton D. M. F, Aquacultural Engineering (2006) 35(3):300-313; Lubian L., 1989, supra).

The present invention provides methods and compositions for flocculating unicellular free-floating microorganisms, for example, microalgae, for example, photosynthetic microalgae. According to present methods, the original culture is an aqueous solution containing free floating microorganismal cells, for example, algae, yeast or bacteria, in a concentration ranging from about 100 to about 2,000 mg/l. In some embodiments, the microorganism (e.g., microalgae) have an average diameter of about 20 μm or less, for example about 15 μm, 10 μm, 5 μm, or less. In some embodiments, the culture contains algal cells of the genus Nannochloropsis, which has been identified as the most difficult marine algal species to be flocculated. See, e.g., Knuckey, et al., supra; and Lubian L., 1989, supra. A suitable amount of aluminum or iron-based coagulant is then added to the culture, providing an intimate and uniform contact between the cells in the culture and the coagulant, for example by gently stirring the culture. The concentration of aluminum or iron-based coagulant is usually 100 mg/l or less, and can vary between 2 and 80 mg/l, for example, about 2 mg/l, 4 mg/l, 5 mg/l, 10 mg/l, 20 mg/l, 30 mg/l, 40 mg/l, 50 mg/l, 60 mg/l, 70 mg/l, 80 mg/l, 90 mg/l or 100 mg/l, resulting in a production cost that enables the commercially viable production of large-volume low-price products.

The aluminum-based coagulants that are effective for this flocculation method include, without limitation, aluminum chloride, aluminum sulfate, polyaluminum chloride, aluminum chlorohydrate, and sodium aluminate. Commercial coagulants are usually solutions characterized by different concentrations of these compounds. For example, commercial aluminum-based coagulants like Tramfloc T552 and T554 (PAC, Poly-Aluminum Chloride) can produce flocculation of the algae Nannochloropsis at a concentration of about 20 mg/l.

Iron-based coagulants effective for this flocculation method include ferric chloride, ferric sulfate, and ferrous sulfate. For example, ferrous sulfate, an inexpensive commodity chemical normally sold as iron sulfate, produces flocculation of the algae Nannochloropsis at a concentration between about 2 mg/l and 100 mg/l, for example, a concentration between 2-80 mg/l, 10-60 mg/l, 5-15 mg/l, 2-10 mg/l, 3-8 mg/l, 4-7 mg/l or 2-5 mg/l. In some embodiments, the ferrous sulfate is present at a concentration of about 2 mg/l, 4 mg/l, 5 mg/l, 10 mg/l, 20 mg/l, 30 mg/l, 40 mg/l, 50 mg/l, 60 mg/l, 70 mg/l, 80 mg/l, 90 mg/l, or 100 mg/l. In some embodiments, the ferrous sulfate is present at a concentration of less than about 10 mg/l, for example less than about 5 mg/l, for example, 10 mg/l, 9 mg/l, 8 mg/l, 7 mg/l, 6 mg/l, 5 mg/l, 4 mg/l, 3 mg/l or 2 mg/l. The concentration can be optimized depending on the salinity and pH of the culture, the removal efficiency desired, and the harvesting or separation method adopted (sedimentation or flotation).

In the present methods, the pH of the aqueous mixture need not be externally adjusted, for example, by the addition of acid or base. The intrinsic or naturally occurring pH will usually be in the range of about 7-10, for example, about pH 7.5-8.5, or about pH 8-10, or about pH 8-9, for example, about 7.0, 7.5, 8.0, 8.5, 9.0, 9.5 or 10.0.

The salinity of the water can be suitable for marine microalgae, and therefore can reflect brackish or sea water. The salinity of the aqueous mixture can be at least about 10 ppt, for example about 10 ppt, 15 ppt, 20 ppt, 25, ppt, 30 ppt, 35 ppt, 36 ppt, 37 ppt, 38 ppt, 39 ppt, or 40 ppt, in some embodiments, 50 ppt or more. In some embodiments, the aqueous mixture has an ionic strength of at least about 0.5, 0.6 or 0.7.

The inorganic coagulant can be in contact with the microorganism culture for at least about 2 minutes (and up to about 15 minutes or longer) to cause the aggregation of the single cells into small flocs. These small flocs can be further aggregated into larger flocs by further adding an organic polyelectrolyte (i.e., polymer) to the culture. The polymer addition to generate larger and heavier flocs allows for convenient harvesting of the microorganisms, for example, by sedimentation. If air flotation or other methods that utilize mechanical forces, including all types of filtration or centrifugation, are employed to remove the flocs instead of gravity, polymer addition can be greatly reduced or even eliminated because larger flocs are not required.

In one embodiment, the flocculation step is followed by gas (air) flotation. A suitable amount of water or culture, typically between about 10% and 30% of the total culture solution that needs to be harvested, is pressurized at a pressure between 20 and 80 psi and saturated with air or other convenient gas. The gas-saturated mixture is released into the culture, creating a bed of bubbles that causes all the floes to float to the surface. Employing the present methods, a clarification efficiency of at least about 75%, for example, at least about 75%, 80%, 85%, 90%, 95% or more, as measured according to the dissolved air flotation assay set forth below, can be reliably achieved. Exposing the culture to dissolved air flotation allows for high harvesting and clarification efficiencies using even lower concentrations of flocculant and/or coagulant, for example flocculant and/or coagulant concentrations of less than about 10 mg/l, or less than about 5 mg/l, for example, about 2 mg/l, 4 mg/l, 5 mg/l or 10 mg/l flocculant and/or coagulant. In some embodiments, a concentration of 4 mg/l ferrous sulfate (FeSO₄) combined with dissolved air flotation can achieve a clarification efficiency of at least 80%.

In one embodiment, the flocculation step is followed by sedimentation. The culture is left in a sedimentation basin or a clarifier until most or all the flocs settle at the bottom of the culture. The basin or the clarifier are designed and built to promote the fastest settling of the flocs, particularly to avoid any parasite or convective flow that would prevent or spoil the natural sedimentation of the algal cells. If sedimentation is utilized as the harvesting method, the inorganic coagulants and the organic polymer can be dosed to produce large floes that can settle with a speed of at least 15 cm/h.

For example, a dosage of 60 mg/l of ferric sulfate and 1 mg/l of organic polymer can achieve the sedimentation of microalgae of the genus Nannochloropsis where more than 70% of the cells display a settling rate higher than 30 cm/h. The settling rate is a measured parameter to define the depth and volume of the clarifier or settling basin. Using the present methods, a sedimentation efficiency of at least about 75%, for example, at least about 75%, 80%, 85%, 90%, 95%, or more, as measured according to the settling velocity measurement assay set forth below, can be reliably achieved in the presence or absence of organic polymer.

In another embodiment, the flocculation step is followed by a combination of sedimentation and gas (air) flotation. In one embodiment, after injection of the inorganic coagulant and the organic polymer, the flocculated culture is sent to a clarifier where, first, the settled biomass is removed from the bottom and, second, air flotation is utilized to float the remaining solids.

In a further aspect, the present methods and compositions provide for the concentration and separation of microalgae of the genus Nannochloropsis by contacting an aqueous culture with a concentration of inorganic flocculant that is less than 10% of the dry biomass of the Nannochloropsis, for example, about 100 mg/l or less, to yield flocs of Nannochloropsis that are at least about 100 μm average diameter; and then separating the floes of Nannochloropsis from the aqueous culture, for example, by sedimentation or air flotation. In some embodiments, the inorganic flocculant is ferrous sulfate or ferric sulfate. In some embodiments, an organic polymer is further added to the aqueous culture. Further embodiments are as described herein.

### EXAMPLES

The following examples are offered to illustrate, but not to limit the claimed invention.

### Methods

Algal cultivation: Cultures of photosynthetic microalgae were maintained in one inch thick Roux flasks with continuous magnetic stirring. Continuous illumination at 700 μE was provided by four 54 watt T12 fluorescent bulbs rated with a correlated color temperature of 5000K. 1% CO₂ was bubbled through sintered glass spargers at a rate sufficient to maintain a pH between 7.0 and 8.5. Photoautotrophic growth was maintained on UFM (Urea Formulated Media), a media formulated with artificial seawater (35 g/L Instant Ocean) containing 720 mg/L urea, 168 mg/L K₂HPO₄, 1.5 ml/L of a metals solution and 1 ml/L of a vitamin solution. The metals solution contained 39.7 g/L Fe(III) Cl₃(6H₂O), 30.0 g/L EDTA, 1.2 g/L MnCl₂(4H₂O), 0.08 g/L CoCl₂(6H₂O), 0.16 g/L ZnSO₄(7H₂O), 0.067 g/L CuSO₄(5H₂O), 0.023 g/L Na₂MoO₄(2H₂O). The vitamins solution contained 0.001 g/L vitamin B12, 0.001 g/L Biotin, and 0.2 g/L Thiamine.

Determination of culture biomass concentration: A sample of the culture between 0.5 and five milliliters was vacuum filtered through a pre-rinsed and pre-ashed Whatman GF/C glass microfiber filter discs. The microalgal cake was rinsed with twenty milliliters of 0.7M ammonium formate and dried for at least 1 hour at 105° C. The dried sample was weighed on an analytical balance and then ashed at 550° C. for at least 1 hour. The post ash weight is subtracted from the pre ash weight and divided by the volume of the sample to get the ash-free dry biomass density in milligrams per milliliter. If the culture was more dense than the experiment calls for then it was diluted with artificial seawater to the appropriate concentration.

Initial Flocculant testing: 10 milliliters of culture were placed into a 10 cm×1.5 cm cylindrical glass tube. Spectrophotometric absorbance at 750 nm was measured for each cell concentration, pH, and salinity condition. The test compound was added and the tube vortexed for 10 seconds. After settling for 30 minutes, a sample was carefully withdrawn from the middle of the tube and absorbance was measured at 750 nm.

Settling velocity measurement: 500 milliliters of culture were placed into a 500 ml graduated cylinder. Spectrophotometric absorbance at 750 nm was measured for each cell concentration, pH, and salinity condition. The test compound was added and the tube was inverted vigorously 5 times. At 30 minute intervals, a sample was carefully withdrawn from the point 15 cm below the meniscus and absorbance was measured at 750 nm.

Dissolved Air Flotation testing: 800 milliliters of culture were placed into a 1000 ml beaker with gentle magnetic stirring. Coagulant was added and stirring continued for several minutes until pin-flocs were visible. If applicable, flocculant was then added and stirring rate adjusted to optimize floc size. The culture was then gently poured into a 1000 ml graduated burette. To prepare dissolved air, 8 liters of artificial seawater were placed into a 10 L pressure vessel with suitable applicator wand. Compressed air was added to bring the pressure in the vessel up to 60 psi (413.7 kPa). The vessel was shaken vigorously for 1 minute, and discharged for 3 seconds to remove any large air bubbles. The applicator wand was used to inject 200 milliliters of dissolved air into the very bottom of the burette containing the coagulated culture. After 5-10 minutes 1 milliliter of solution was withdrawn from the burette stopcock and absorbance was measured at 750 nm.

### Example 1

This Example demonstrates the successful concentration and separation of microalgae of the genus Nannochloropsis by first flocculating the microalgae with low concentrations of inorganic flocculant and then sedimenting the microalgae.

The inorganic coagulant—e.g., Fe- or Al-based—was dissolved in water at a concentration of 10 g/L. Vigorous stirring was required with Fe-based coagulants but, eventually, all the inorganic coagulants were completely soluble in water at the above concentration. The organic polyelectrolyte—for example, Tramfloc T141, Zetag 8818, Praestol K₂₉₀FL, Monolyte 6016—was dissolved in water at a concentration of 1 ml/L. This also required vigorous agitation, but it dissolved fairly quickly.

First, the inorganic coagulant solution was added to the Nannochloropsis microalgae culture having a biomass density of 250 mg/l. This was agitated vigorously for 30 seconds and then stirred more gently until small flocs were clearly visible. This required up to 10 minutes, depending on the amount of coagulant injected into the culture. Second, the organic polymer solution was added to the coagulated culture. Agitation was increased enough to completely disperse the polymer, and then slowed enough to allow flocs to aggregate. The polymer acted very quickly and within 2 minutes, aggregation of the small flocs into larger flocs was clearly visible. The larger the polymer dose (up to 3 mg/l), the larger and heavier the aggregated flocs were, which eventually resulted in faster and more efficient sedimentation.

Two parameters were used to judge the performance of the sedimentation-based harvesting process: (1) settling speed (i.e., how fast the algae flocs settled to the bottom of the culture) and (2) removal efficiency (i.e., what portion of the algae flocs were eventually removed from the culture). For example, a culture having a biomass density of 250 mg/l (dry basis), 80 mg/l of inorganic coagulant and 2 mg/l of organic polymer was sufficient for complete water clarification and biomass separation.

A summary of the results obtained with this procedure is here shown in Tables 1-3: Salinity and pH of the algae culture affected the doses of coagulants and organic polymers required for a desired sedimentation efficiency.

TABLE 1
Aluminum-Based Coagulants, Salinity = 35 ppt, pH = 9.2

| Inorganic Coagulant | Organic Polymer | Sedimentation Efficiency |
|---|---|---|
| 100 mg/l Al Chloride | 2 mg/l | 72.2% |
| 100 mg/l Al Sulfate | 2 mg/l | 74.6% |
| 100 mg/l Alum | 2 mg/l | 62.2% |
| 100 mg/l PAC | 2 mg/l | 0.2% |

TABLE 2
Aluminum-Based Coagulants, Salinity = 17 ppt, pH = 10.0

| Inorganic Coagulant | Organic Polymer | Sedimentation Efficiency |
|---|---|---|
| 20 mg/l PAC | — | 93.0% |

TABLE 3
Iron-Based Coagulants, Salinity = 35 ppt, pH = 9.6

| Inorganic Coagulant | Organic Polymer | Sedimentation Efficiency |
|---|---|---|
| 100 mg/l Ferric Sulfate | 2 mg/l | 93.7% |
| 80 mg/l Ferric Sulfate | 2 mg/l | 92.8% |
| 60 mg/l Ferric Sulfate | 2 mg/l | 85.8% |
| 40 mg/l Ferric Sulfate | 2 mg/l | 74.9% |
| 100 mg/l Ferric Sulfate | — | 86.3% |
| 80 mg/l Ferric Sulfate | — | 78.2% |
| 60 mg/l Ferric Sulfate | — | 54.4% |
| 40 mg/l Ferric Sulfate | — | 21.8% |
| 100 mg/l Ferrous Sulfate | 2 mg/l | 90.9% |
| 80 mg/l Ferrous Sulfate | 2 mg/l | 90.1% |
| 60 mg/l Ferrous Sulfate | 2 mg/l | 84.5% |
| 40 mg/l Ferrous Sulfate | 2 mg/l | 66.0% |
| 100 mg/l Ferrous Sulfate | — | 78.3% |
| 80 mg/l Ferrous Sulfate | — | 77.0% |
| 60 mg/l Ferrous Sulfate | — | 60.4% |
| 40 mg/l Ferrous Sulfate | — | 21.2% |

### Example 2

This Example demonstrates the successful concentration and separation of microalgae of the genus Nannochloropsis by first flocculating the microalgae with low concentrations of inorganic flocculant and then further concentration of the microalgae by air flotation.

The initial flocculation step were performed similarly to the procedures described in the previous example. The inorganic coagulant—e.g., Fe- or Al-based—was dissolved in water at a concentration of 10 g/L and the organic polymer was dissolved in water at a concentration of 1 ml/L.

The inorganic coagulant solution was first added to the Nannochloropsis microalgae culture having a biomass density of 250 mg/l. This was agitated vigorously for 30 seconds and then stirred more gently until small flocs were clearly visible. This required up to 10 minutes, depending on the amount of coagulant injected into the culture. Second, the organic polymer solution was added to the coagulated culture. Agitation was increased enough to completely disperse the polymer, and then slowed enough to allow flocs to aggregate. The polymer acted very quickly and within up to 2 minutes, aggregation of smaller flocs into larger flocs was clearly visible. The concentrations of coagulant and polymer required for biomass removal with air flotation were lower than those required for sedimentation.

Dissolved air flotation. 800 milliliters of the coagulated culture were gently poured into a 1000 ml graduated burette. To prepare dissolved air, 8 liters of artificial seawater were placed into a 10 L pressure vessel with suitable applicator wand. Compressed air was added to bring the pressure in the vessel up to 60 psi (413.7 kPa). The vessel was shaken vigorously for 1 minute, and discharged for 3 seconds to remove any large air bubbles. The applicator wand was used to inject 200 milliliters of dissolved air into the very bottom of the burette containing the coagulated culture. After 5-10 minutes, 1 milliliter of solution was withdrawn from the burette stopcock and absorbance was measured at 750 nm.

The degree of clarification of the culture or, conversely, the biomass removal efficiency were the measured performance parameters for the flotation-based harvesting process. For example, with a culture having a biomass density of 250 mg/L (dry basis), 40 ppm of inorganic coagulant and 0.25 ppm of organic polymer were sufficient for substantial water clarification and biomass separation.

TABLE 4
Aluminum-Based Coagulants, Salinity = 35 ppt, pH = 9.2

| | | Clarification |
|---|---|---|
| Inorganic Coagulant | Organic Polymer | Efficiency |
| 50 mg/l Aluminum Chloride | 0.25 mg/l | 85.8% |
| 20 mg/l PAC | 0.25 mg/l | 70.0% |
| 50 mg/l Alum | 0.25 mg/l | 70.4% |
| 50 mg/l Aluminum Sulfate | 0.25 mg/l | 70.1% |

TABLE 5
Iron-Based Coagulants, Salinity = 35 ppt, pH = 8.3

| | | |
|---|---|---|
| | | Clarification |
| Inorganic Coagulant | Organic Polymer | Efficiency |

| | | | |
|---|---|---|---|
| 50 mg/l Ferric Sulfate | 0.25 | mg/l | 85.4% |
| 40 mg/l Ferric Sulfate | 0.25 | mg/l | 80.2% |
| 30 mg/l Ferric Sulfate | 0.25 | mg/l | 68.3% |
| 50 mg/l Ferrous Sulfate | 0.25 | mg/l | 89.2% |
| 40 mg/l Ferrous Sulfate | 0.25 | mg/l | 87.7% |
| 30 mg/l Ferrous Sulfate | 0.25 | mg/l | 79.7% |
| 50 mg/l Ferrous Sulfate | 1 | mg/l | 90.8% |
| 50 mg/l Ferric Chloride | 0.25 | mg/l | 86.4% |
| 40 mg/l Ferric Chloride | 0.25 | mg/l | 81.7% |
| 30 mg/l Ferric Chloride | 0.25 | mg/l | 68.9% |
| 50 mg/l Ferric Chloride | 1 | mg/l | 89.4% |

### Example 3

This Example shows the successful scale-up for dissolved air flotation (DAF) harvesting of microalgae.

Microalgae can be separated from aqueous solution by treatment with flocculants, coagulants, and polymers or a combination of these inorganic additions and applying micro-bubbles to the liquid column to float the flocculated particles out of solution. At the pilot or laboratory scale, this was performed using a graduated cylinder and an air stone capable of producing sufficiently small bubbles.

A commercial dissolved air flotation unit was employed to demonstrate the process on a larger scale. The equipment utilized had a maximum hydraulic capacity of 60 gallons per minute (gpm), and a flow rate of 15-16 gpm was utilized for the testing. A solution of FeSO₄ was used as a flocculant resulting in an iron (Fe) concentration of approximately 4 mg/l. The subsequent mixture was delivered to the influent line of the DAF equipment. Micro-bubbles were generated by the DAF onboard unit using recycled clarified effluent from the system at a rate of approximately 25% (3-4 gpm) compared to the incoming untreated effluent.

Information from the DAF harvesting is listed below. All tests were performed with a Nannochloropsis culture cultivated in open ponds. Harvesting of the ponds was not performed on a regular schedule, thus non-consecutive dates are represented in Table 6.

TABLE 6

| | Composite Untreated | DAF Clarified Effluent | DAF Concentrate | |
|---|---|---|---|---|
| | AFDW | AFDW | AFDW | DAF Clarifying |
| Day# | (mg/l) | (mg/l) | (mg/l) | Efficiency % |
| 21 | 150 | 15 | 20150 | 90 |
| 32 | 115 | 20 | 13412 | 83 |
| 36 | 78 | 16 | 6266 | 79 |
| 37 | 106 | 23 | 8587 | 78 |
| 40 | 163 | 32 | 13357 | 80 |
| 42 | 164 | 30 | 12454 | 82 |
| 43 | 137 | 30 | 11507 | 78 |
| 44 | 133 | 26 | 7396 | 80 |
| 54 | 188 | 27 | 15559 | 86 |
| 56 | 156 | 29 | 11910 | 81 |
| 58 | 151 | 27 | 8210 | 82 |
| 63 | 193 | 38 | 14310 | 80 |
| 65 | 153 | 31 | 12095 | 80 |
| 69 | 210 | 40 | 21505 | 81 |
| 70 | 210 | 47 | 17480 | 78 |

The results of Table 6 show that harvesting efficiencies remained consistently above 80%, thus proving the mechanical viability of this method in separating microalgae from solution.

### Example 4

This Example shows the successful scaling-up for settling of microalgae.

Microalgae can be separated from aqueous solution by settling after treatment with flocculants, coagulants, and polymers or a combination of these inorganic additions. At the pilot or laboratory scale, this was performed using a graduated cylinder and measuring the settling speed and final clarification of the aqueous medium.

The same method was demonstrated at a larger scale utilizing a conical tank with a capacity of 378 liters. A solution of FeCl₃ was used as a coagulant resulting in an iron (Fe) concentration of approximately 7 mg/l. A solution of Tramfloc 141 polyacrylamide emulsion was used as organic polymer. Both the coagulant and the polymer were added to the algal culture in the conical tank and properly mixed. The resulting flocculated culture was left in the conical tank for 0.5 to 1.0 hours to settle.

TABLE 7
Data from the settling of Nannochloropsis
cultures cultivated in open ponds^{§}

| Initial | | | |
|---|---|---|---|
| biomass | Polymer | Biomass | Harvesting |
| density | addition | recovered | efficiency |
| (mg/l) | (ml) | (grams) | (%) |
| 204 | 200 | 31.0 | 84.5% |
| 181 | 200 | 29.8 | 91.4% |
| 177 | 200 | 28.6 | 89.9% |
| 134 | 200 | 23.2 | 96.3% |
| 151 | 200 | 27.1 | 99.8% |
| 138 | 200 | 22.5 | 90.8% |
| 232 | 250 | 37.4 | 89.7% |
| 221 | 200 | 38.7 | 97.2% |
| 212 | 200 | 36.4 | 95.4% |
| 226 | 200 | 35.7 | 87.8% |
| 220 | 200 | 35.7 | 90.1% |
| 210 | 200 | 34.7 | 91.9% |
| 214 | 250 | 36.7 | 95.3% |
| 199 | 200 | 32.6 | 91.1% |
| 217 | 200 | 36.4 | 93.2% |
| 195 | 200 | 32.9 | 93.8% |
| 168 | 250 | 27.8 | 92.1% |
| 182 | 200 | 29.1 | 88.9% |
| 230 | 250 | 39.1 | 94.4% |
| 237 | 200 | 37.1 | 87.1% |
| 233 | 250 | 38.1 | 90.9% |
| 223 | 200 | 38.4 | 95.7% |
| 243 | 250 | 37.3 | 85.3% |
| 219 | 200 | 38.2 | 96.7% |

^{§}Culture volume was 180 liters. 1 liter of coagulant was added.

The results of Table 7 show that harvesting efficiencies remained consistently above 80% with the specified coagulant and polymer concentrations, proving the technical viability of the present separation methods. Efficiency was measured based the comparison of the untreated samples or initial biomass measurements of the volume treated and the biomass recovered.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of concentrating single cell microalgae in an aqueous environment, the method comprising:
a) contacting microalgae having an average single cell diameter of less than 20 μm in an aqueous environment with an inorganic flocculant present at a concentration that is less than 10% of the dry biomass of the microalgae, thereby yielding flocculated microalgae in flocs having an average diameter of at least 100 μm; and
b) separating the flocs of microalgae from the aqueous environment, thereby concentrating the microalgae into a slurry with a biomass density of at least 1%.

2. The method of claim 1, wherein the inorganic coagulant is present at a concentration of 100 mg/l or less.

3. The method of claim 1, wherein the flocculant is present at a concentration between 2 mg/l and 80 mg/l.

4. The method of claim 1, wherein the flocculant is present at a concentration between 2 mg/l and 10 mg/l.

5. The method of claim 1, wherein the flocculant is an iron flocculant or an aluminum flocculant.

6. The method of claim 5, wherein the flocculant is an aluminum flocculant selected from the group consisting of aluminum chloride, aluminum sulfate, polyaluminum chloride, aluminum chlorohydrate, and sodium aluminate.

7. The method of claim 5, wherein the flocculant is an iron flocculant selected from the group consisting of ferric chloride, ferric sulfate, and ferrous sulfate.

8. The method of claim 1, wherein the flocculant is not algicidal.

9. The method of claim 1, wherein the microalgae are in a non-natural body of water.

10. The method of claim 1, wherein the microalgae in the aqueous environment are essentially a monoculture.

11. The method of claim 1, wherein the flocs of microalgae are separated from the aqueous environment to produce a slurry with a biomass density of 1-10%.

12. The method of claim 1, wherein the separating step comprises subjecting the flocculated algae to air flotation.

13. The method of claim 1, wherein the separating step comprises subjecting the flocculated algae to sedimentation.

14. The method of claim 1, wherein the microalgae has an average single cell diameter of less than 10 μm.

15. The method of claim 1, wherein the microalgae has an average single cell diameter of less than 5 μm.

16. The method of claim 1, wherein the microalgae is from a microalgal strain selected from the group consisting of Dunaliella, Chlorella, Tetraselmis, Botryococcus, Haematococcus, Phaeodactylum, Skeletonema, Chaetoceros, Isochrysis, Nannochloropsis, Nannochloris, Pavlova, Nitzschia, Pleurochrysis, Chlamydomas and Synechocystis.

17. The method of claim 16, wherein the microalgae is Nannochloropsis.

18. The method of claim 1, further comprising contacting the microalgae with an organic polymer.

19. The method of claim 18, wherein the organic polymer is a cationic or a non-ionic polymer.

20. The method of claim 18, wherein the organic polymer is comprised of monomers selected from the group consisting of acrylamide, acrylate, amine or mixtures thereof.

21. The method of claim 18, wherein the organic polymer is from a naturally occurring source.

22. The method of claim 21, wherein the organic polymer is chitosan or a clay.

23. The method of claim 18, wherein the organic polymer is present in a concentration of less than 2% of the weight of the dry biomass.

24. The method of claim 1, wherein the aqueous environment is free of sewage.

25. The method of claim 1, wherein the aqueous environment is free of polybasic carboxylic acid.

26. The method of claim 1, wherein the aqueous environment contains only trace amounts of copper.

27. The method of claim 1, wherein the aqueous environment is less than pH 10.

28. The method of claim 27, wherein the aqueous environment is between pH 7-9.

29. The method of claim 27, wherein the aqueous environment is not externally pH adjusted.

30. The method of claim 1, wherein the aqueous environment has a salinity of at least 20 ppt.
